(19) United States
(12) Patent Application Publication
(10) Pub. No.: US 2024/0299007 A1
(43) Pub. Date: Sep. 12, 2024

(54) ADHESIVE HYDROPHILIC PAD FOR ULTRASOUND TRANSDUCER

(71) Applicant: CIVCO Medical Instruments Co., Inc., Kalona, IA (US)

(72) Inventors: Geoffrey Scott Wagner, Fairfax, IA (US); Gregory Nephi Nordgren, North Liberty, IA (US); Ryan Albert Swartz, Coralville, IA (US)

(21) Appl. No.: 18/670,173

(22) Filed: May 21, 2024

Related U.S. Application Data
(63) Continuation of application No. 16/139,625, filed on Sep. 24, 2018.
(60) Provisional application No. 62/565,736, filed on Sep. 29, 2017.

(51) Int. Cl. A61B 8/00; C08J 7/04; C08J 7/043; C08J 7/056

(52) U.S. Cl. CPC A61B 8/4236; A61B 8/4281; C08J 7/0427; C08J 7/043; C08J 7/056; A61B 8/4422; C08J 2375/04; C08J 2433/12; C08J 2439/06; C08J 2475/04

(57) ABSTRACT

A disposable ultrasound transducer interface pad configured to adhere to at least one of an operational portion of an ultrasound transducer or a skin surface of a patient, includes an impermeable substrate layer having a first surface and a second surface opposite the first surface. A first hydrophilic coating layer is applied to the first surface of the impermeable substrate layer. The first hydrophilic coating layer is activatable to provide an acoustic coupling interface between the ultrasound transducer and the patient. An adhesive layer is formed on the second surface of the impermeable substrate layer and configured to adhere to the operational portion of the ultrasound transducer or the skin surface of the patient.

### CROSS-REFERENCE TO RELATED APPLICATIONS

This application is a continuation of prior application Ser. No. 16/139,625, filed Sep. 24, 2018, which claims the benefit of U.S. Provisional Application No. 62/565,736, filed on Sep. 29, 2017, the entirety of both applications are hereby incorporated by reference herein.

### BACKGROUND

This invention relates to medical devices and more particularly to ultrasound transducers and devices for covering the ultrasound transducer for use in external, intraoperative, or endocavity applications.

Ultrasound transducers are commonly used in clean, but non-sterile environments, such as patient examination rooms. In many typical ultrasound procedures, such as prenatal abdominal ultrasounds, bladder or other organ screenings, etc., an acoustic ultrasound gel is applied to a supine patient's abdomen and an ultrasound transducer is positioned to contact the gel and is moved around the abdomen to acquire ultrasound images. Once the procedure is complete, both the patient and the ultrasound transducer must be cleaned of gel. In circumstances in which time between procedures is a concern, the cleaning process negatively impacts productivity.

### BRIEF DESCRIPTION OF THE DRAWINGS

FIG. 1A-1C illustrate an environment in which embodiments described herein may be implemented;

FIGS. 2A-2C are cross-sectional views of an exemplary implementation of the interface pad of FIG. 1; and

FIGS. 3A-3C are flow charts illustrating exemplary processes of forming and using an ultrasound transducer interface pad in accordance with embodiments described herein.

### DETAILED DESCRIPTION OF THE PREFERRED EMBODIMENTS

The following detailed description refers to the accompanying drawings. The same reference numbers in different drawings may identify the same or similar elements. Also, the following detailed description does not limit the invention.

Implementations described herein relate to materials for providing an effective and easy to use interface between an ultrasound transducer and a patient. Consistent with one implementation described herein, a disposable transducer interface includes a multi-layer configuration, hereinafter referred to as a “pad,” for engaging an operating end of the transducer on one side and a patient's skin on the opposite side. In one embodiment, the multi-layer interface pad includes a carrier layer, with an adhesive layer and a hydrophilic layer applied to opposing sides of the carrier layer. During use, the adhesive layer side of the pad removably adheres to the transducer (or patient) to provide a positive, consistent coupling between the pad and the transducer. The hydrophilic layer is then hydrated to provide a positive acoustic coupling that facilitates clear and efficient transmission of ultrasound signals therethrough and eliminates the need to use traditional acoustic coupling gel.

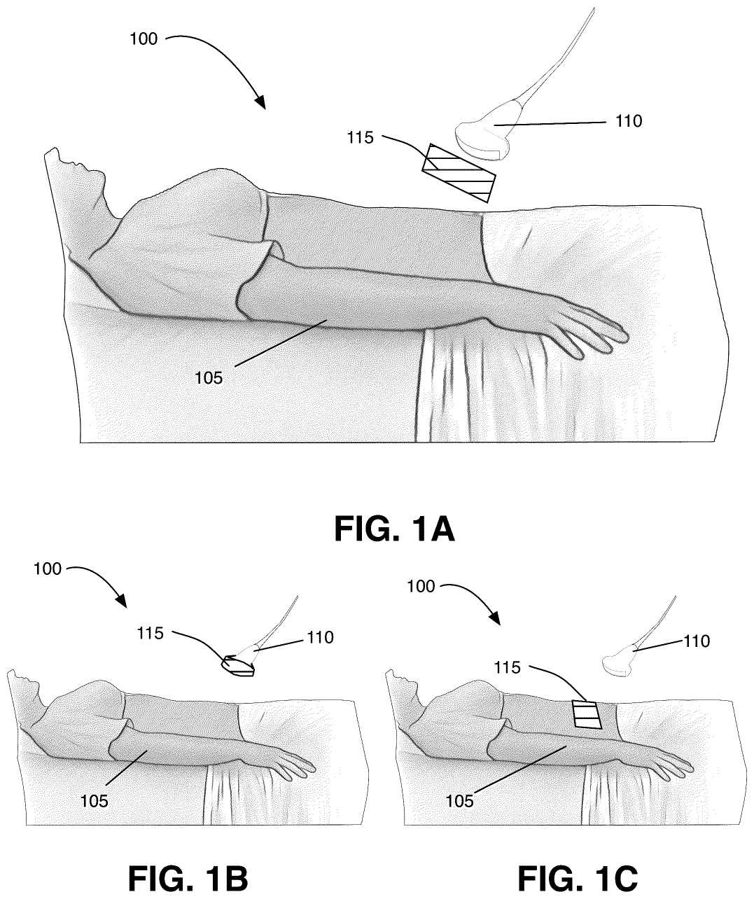

FIG. 1A-1C illustrate an environment 100 in which embodiments described herein may be implemented. As shown in FIG. 1A, environment 100 includes a patient 105, an ultrasound transducer 110, and an interface pad 115. During use, as shown in FIGS. 1B and 1C, interface pad 115 may be used in one of two manners. In a first embodiment, as shown in FIG. 1B, interface pad 115 may be adhered, as described below, to the operational end of ultrasound transducer 110, while in the second embodiment, as shown in FIG. 1C, interface pad 115 may be adhered to patient 105. In either embodiment, once affixed to either transducer 110 or patient 105, pad 115 may be hydrated by applying a liquid, such as water, saline, lidocaine, chloraprep, isopropyl alcohol, or other like solution to the exposed surface to form an acoustically efficient interface and allow for easy movement (i.e., sliding) between transducer 110 and interface pad 115. In some embodiments, a patient's bodily fluid or excretions may be sufficient to hydrate pad 115. In such embodiments, external or added hydrating solutions may not be necessary.

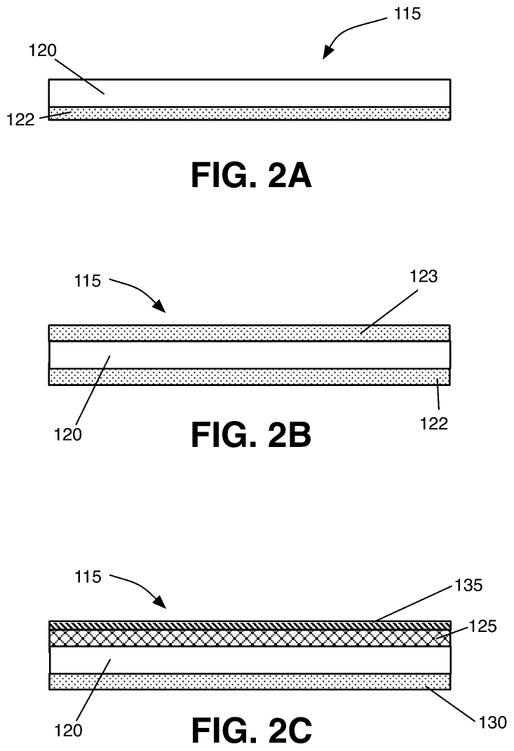

FIG. 2A-2C illustrate cross-sectional views of exemplary implementations of interface pad 115. As shown in FIG. 2A, interface pad 110 includes a substrate layer 120, such as a polyurethane carrier or material having a thickness ranging from approximately 0.025 to 1.0 mm. Consistent with implementations described herein, substrate layer 120 may be formed in either a planar or non-planar (e.g., shaped) configuration depending on application. For example, in some embodiments, substrate layer 120 may have a shaped (e.g., three-dimensional) configuration corresponding to the ultrasound transducer onto which it is to be applied. In other embodiments, substrate layer 120 may be formed as a planar layer usable with a number of different transducers and in a variety of procedures.

Consistent with embodiments described herein, interface pad 115 further includes a hydrophilic coating layer 122 applied to one side of substrate layer 120. In this configuration, hydrophilic coating layer 122 is provided on an outside of interface pad 115 relative to transducer 110.

In one embodiment, hydrophilic coating layer 122 includes an ultra-violet (UV) light or heat curable materials, such as polyvinylpyrrolidone/polyurethane (PVP/PU) or poly methacrylate (PM), having a thickness in the range of approximately 2 to 5 microns. During manufacture, hydrophilic coating layer 122 may be applied to the substrate layer 120 and cured via exposure to UV light or exposing the layer to heat.

During use, an acoustic coupling gel may be applied to an inside of substrate layer 120 prior to applying interface pad 115 to the ultrasound probe. Next, hydrophilic coating layer 122 may be activated using only water or saline to provide the requisite acoustic coupling interface between transducer 110 and patient 105.

Consistent with embodiments described herein, interface pad 115 may be formed of any suitable shape or dimensions consistent with the particular ultrasound transducer or patient body part with which it is to be used. For example, in one embodiment, interface pad 115 may be formed in a rectangular configuration having a length of approximately 5 inches and a width of approximately 3.25 inches.

FIG. 2B illustrates an embodiment of interface pad 115 that includes a second hydrophilic coating layer 123 applied on a side of substrate layer 120 opposite from hydrophilic coating layer 122. During use, hydrophilic coating layers 122 and 123 may each be activated using only water or saline to provide the requisite acoustic coupling interface between transducer 110 and patient 105.

In another implementation, as shown in FIG. 2C, interface pad 115 includes substrate layer 120, an adhesive layer 125, a hydrophilic coating layer 130, and a removable release layer 135. In one embodiment, substrate layer 120 comprises a polyurethane film carrier or material, such as polyether polyurethane having a thickness ranging from approximately 0.025 to 1.00 millimeters (mm).

In some implementations, adhesive layer 125 may include a silicone-based adhesive, having, for example, an adhesion (or removal force) of between 0.2 and 0.8 Newtons (N) per 25 millimeters (mm). The relatively low removal force of such a silicon-based adhesive renders interface pad 115 generally repositionable after initial deployment. Furthermore, such silicone-based adhesives are capable of sticking to itself without destroying the product during initial deployment, repositioning or removing.

In other embodiments, adhesive layer 125 may include an acrylic or synthetic rubber-based adhesive material. Such non-silicone-based adhesives, may exhibit significantly higher removal forces (e.g., as high as 16.7N per 25 mm). An adhesive having a higher removal force may be desirable in some circumstances, such as where slippage of the pad during use is a concern.

Consistent with embodiments described herein, adhesive layer 125 is applied (e.g., coated) onto substrate layer 120 at a coat weight ranging from approximately 100 to 200 grams per square meter (gsm), and preferably at a coat weight of 150 gsm, resulting in adhesive layer 125 having an applied thickness ranging from 0.025 to 0.2 mm (e.g., 0.15 mm).

As shown in FIG. 2C, hydrophilic coating layer 130 is applied to substrate layer 120 on an opposite side of substrate layer 120 relative to adhesive layer 125. During manufacture and prior to use, interface pad 115 includes a release layer 135 (also referred to as a liner or release liner) that is provided on adhesive layer 125 to protect the tackiness of adhesive layer 125 and to prevent adhesive layer 125 from adhering to other items or itself prior to use. In one implementation, release layer 135 comprises a polycarbonate layer. Consistent with embodiments described herein, release layer 135 is removed (e.g., peeled off) prior to using interface pad 115, e.g., prior to adhering interface pad 115 to transducer 110/patient 105. In some embodiments, release layer 135 may include an edge area or slit that allows release layer 135 to be easily removed when interface pad 115 is ready for use.

Although interface pad 115 of FIG. 2C is described above as including three distinct layers 120-130 and a release layer 135, in other implementations, interface pad 115 may be formed of only two layers, with an adhesive layer 125 being applied directly to hydrophilic coating layer 130, without the requirement of an underlying polyurethane substrate layer.

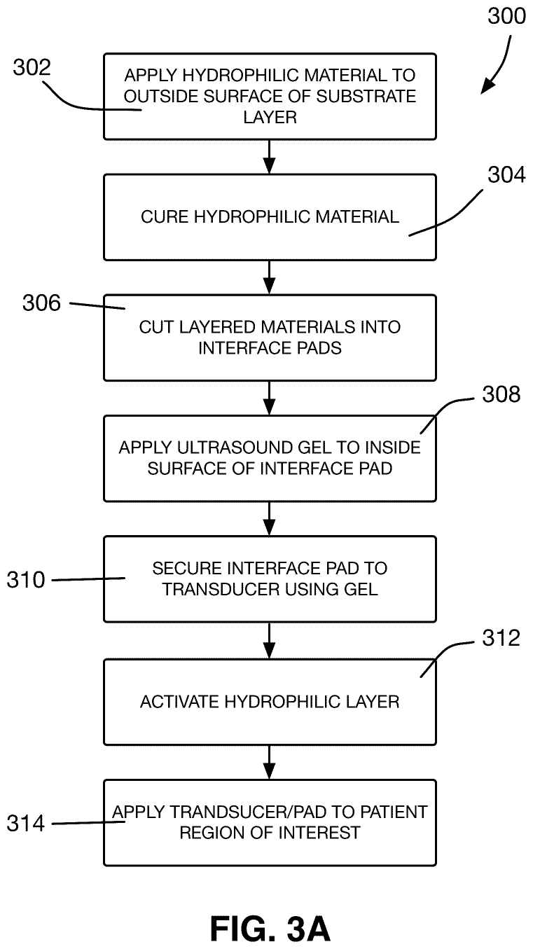

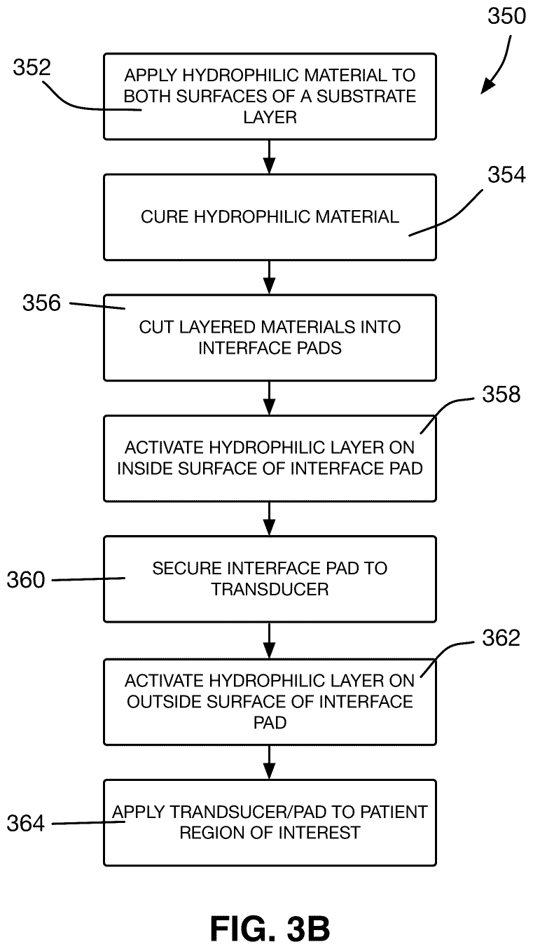

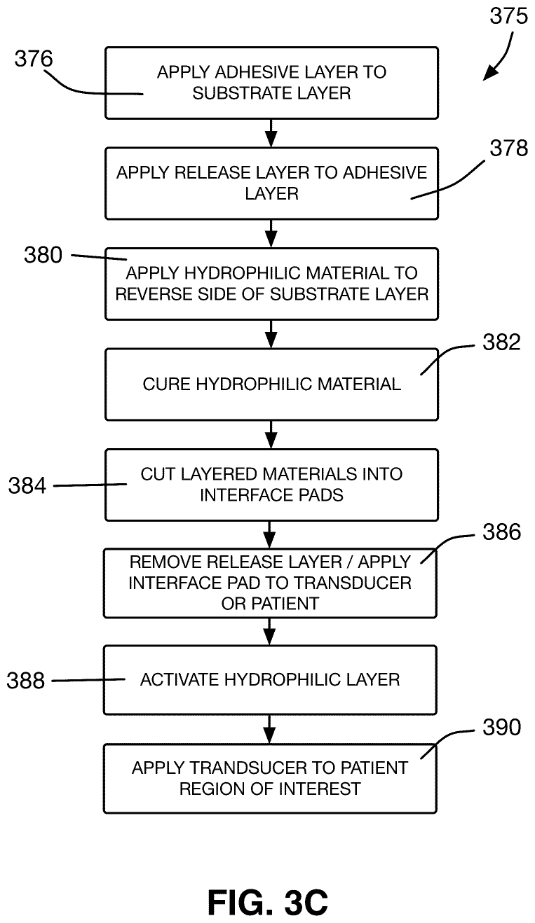

FIGS. 3A-3C illustrate flow charts illustrating exemplary processes 300, 350, and 375, respectively of forming and using an ultrasound transducer interface pad in accordance with embodiments described herein, with process 300 corresponding to the embodiment of FIG. 2A, process 350 corresponding to the embodiment of FIG. 2B. and process 375 corresponding to the embodiment of FIG. 2C.

Referring to FIG. 3A, a hydrophilic material may be coated onto one side of a substrate material (block 302). For example, hydrophilic layer 122 may be coated (e.g., sprayed, brushed, etc.) on a side of a sheet of polyurethane substrate. At block 304, the hydrophilic material is cured, such as via heat or UV light. At block 306, one or more interface pads 115 are cut to a desired size and/or shape, such as with a die cut machine.

At block 308, a traditional ultrasound coupling gel is applied to an inside surface of an interface pad 115. Next, interface pad 115 is applied to an operating end of ultrasound transducer 110 to secure interface pad 115 to transducer 110 (block 310). Next, the hydrophilic layer is activated (block 312). For example, a water or saline may be applied to hydrophilic layer 122. Finally, the ultrasound transducer with the activated interface pad secured thereto is applied to a region of interest on a patient (block 314).

Referring to FIG. 3B, a hydrophilic material may be coated onto both sides of a substrate material (block 352). For example, hydrophilic layers 122 and 123 may be coated (e.g., sprayed, brushed, etc.) onto the sides of a sheet of polyurethane substrate. At block 354, the hydrophilic material is cured, such as via heat or UV light. In some implementations, hydrophilic layer application and curing are done independently for each of layers 122 and 123. At block 356, one or more interface pads 115 are cut to a desired size and/or shape, such as with a die cut machine.

At block 358, one of the hydrophilic layers is activated. For example, hydrophilic layer 123 is activated using water or saline. Next, interface pad 115 is applied to an operating end of ultrasound transducer 110 to secure interface pad 115 to transducer 110 via the activated hydrophilic layer 123 (block 360). Next, the other hydrophilic layer is activated (block 362). For example, a water or saline may be applied to hydrophilic layer 122. Finally, the ultrasound transducer with the activated interface pad secured thereto is applied to a region of interest on a patient (block 364).

Referring to FIG. 3C, an adhesive layer is initially applied to a substrate layer (block 376). For example, a silicone adhesive material may be coated (e.g., poured, sprayed, brushed, etc.) onto a first surface of a polyurethane substrate layer, such as substrate layer 120 to form the adhesive layer. Next, a release layer, such as a polymeric or paper layer, may be applied to the adhesive layer to prevent the adhesive from losing tackiness or sticking to unintended materials (block 378). For example, release layer 135 may be applied to a tacky side of adhesive layer 125.

Next, a hydrophilic material may be coated on a reverse side of the substrate layer (block 380). For example, hydrophilic layer 130 may be coated (e.g., poured, sprayed, brushed, etc.) on a side of substrate 120 opposite to adhesive layer 125. At block 382, the hydrophilic material is cured, such as via heat or UV light. At block 384, one or more interface pads are cut to a desired size and/or shape from the layered materials, such as with a die cut machine.

At block 386, the release layer is removed, and the adhesive layer is applied to either to an operating end of an ultrasound transducer or directly to the region of interest on the patient. For example, release layer 135 is removed to expose the tacky side of adhesive layer 125 and the adhesive layer 125 is then applied to transducer 110 or patient 105. Next, at block 388, the hydrophilic layer is activated. For example, hydrophilic layer 130 is activated using water or saline. Finally, either the ultrasound transducer with the activated interface pad secured thereto is applied to a region of interest on a patient or the ultrasound transducer is applied to the activated interface pad secured to the patient (block 390).

Consistent with embodiments described herein, the interface pad may be packaged as either a sterile or a non-sterile product for use in different medical environments or circumstances.

The foregoing description of exemplary implementations provides illustration and description but is not intended to be exhaustive or to limit the embodiments described herein to the precise form disclosed. Modifications and variations are possible in light of the above teachings or may be acquired from practice of the embodiments.

Although the invention has been described in detail above, it is expressly understood that it will be apparent to persons skilled in the relevant art that the invention may be modified without departing from the spirit of the invention. Various changes of form, design, or arrangement may be made to the invention without departing from the spirit and scope of the invention. Therefore, the above-mentioned description is to be considered exemplary, rather than limiting, and the true scope of the invention is that defined in the following claims.

No element, act, or instruction used in the description of the present application should be construed as critical or essential to the invention unless explicitly described as such. Also, as used herein, the article “a” is intended to include one or more items. Further, the phrase “based on” is intended to mean “based, at least in part, on” unless explicitly stated otherwise.

Use of ordinal terms such as “first,” “second,” “third,” etc., in the claims to modify a claim element does not by itself connote any priority, precedence, or order of one claim element over another, the temporal order in which acts of a method are performed, the temporal order in which instructions executed by a device are performed, etc., but are used merely as labels to distinguish one claim element having a certain name from another element having a same name (but for use of the ordinal term) to distinguish the claim elements.

What is claimed is:

1. A disposable ultrasound transducer interface pad configured to adhere to at least one of an operational portion of an ultrasound transducer or a skin surface of a patient, the ultrasound transducer interface pad comprising:
an impermeable substrate layer having a first surface and a second surface opposite the first surface;
a first hydrophilic coating layer applied to the first surface of the impermeable substrate layer, the first hydrophilic coating layer activatable to provide an acoustic coupling interface between the ultrasound transducer and the patient; and
an adhesive layer formed on the second surface of the impermeable substrate layer and configured to adhere to the operational portion of the ultrasound transducer or the skin surface of the patient.

2. The disposable ultrasound transducer interface pad of claim 1, wherein the impermeable substrate layer comprises a polyurethane film material.

3. The disposable ultrasound transducer interface pad of claim 1, wherein the impermeable substrate layer has a thickness of 0.025 millimeter (mm) to 1.00 mm.

4. The disposable ultrasound transducer interface pad of claim 3, wherein the first hydrophilic coating layer has a thickness of 0.002 millimeter (mm) to 0.005 mm.

5. The disposable ultrasound transducer interface pad of claim 4, wherein the adhesive layer has a thickness of 0.025 millimeter (mm) to 0.2 mm.

6. The disposable ultrasound transducer interface pad of claim 1, wherein the impermeable substrate layer is configured to prevent particulates greater than 27 nanometers from penetrating the impermeable substrate layer according to ASTM F1671/F1671M-22.

7. The disposable ultrasound transducer interface pad of claim 1, further comprising a release layer provided on the adhesive layer.

8. The disposable ultrasound transducer interface pad of claim 1, wherein the first hydrophilic coating layer is configured to be activatable by a liquid.

9. The disposable ultrasound transducer interface pad of claim 1, wherein the impermeable substrate layer comprises polyurethane, the impermeable substrate layer having a thickness of 0.025 millimeter (mm) to 1.0 mm.

10. The disposable ultrasound transducer interface pad of claim 1, wherein the adhesive layer comprises a silicone adhesive material.

11. The disposable ultrasound transducer interface pad of claim 10, wherein the silicone adhesive material has a thickness ranging from 0.025 millimeter (mm) to 0.2 mm.

12. The disposable ultrasound transducer interface pad of claim 10, wherein the silicone adhesive material has a coat weight in a range of 100 grams per square meter (gsm) to 200 gsm.

13. The disposable ultrasound transducer interface pad of claim 10, wherein the silicone adhesive material has an adhesion of between 0.2 Newtons (N) per 25 millimeters (mm) and 0.8 N per 25 mm.

14. The disposable ultrasound transducer interface pad of claim 1, wherein the first hydrophilic material comprises one of an ultraviolet (UV) light curable hydrophilic material or a heat curable hydrophilic material.

15. The disposable ultrasound transducer interface pad of claim 1, wherein the first hydrophilic coating layer comprises polyvinylpyrrolidone/polyurethane or poly methacrylate, the first hydrophilic coating layer having a thickness of 2.0 microns to 5.0 microns.

16. The disposable ultrasound transducer interface pad of claim 1, further comprising a removable release layer coupled to the adhesive layer, the removable release layer configured to be removed from the adhesive layer to expose the adhesive layer for adhering the disposable ultrasound transducer interface pad to the operational portion of the ultrasound transducer or the skin surface of the patient.

17. The disposable ultrasound transducer interface pad of claim 1, wherein the impermeable substrate layer is made of a flexible material.

18. The disposable ultrasound transducer interface pad of claim 1, further comprising a second hydrophilic coating layer applied to the second surface of the impermeable substrate layer, the second hydrophilic coating layer activatable to provide an additional acoustic coupling interface between the ultrasound transducer and the patient.

19. The disposable ultrasound transducer interface pad of claim 1, wherein the first hydrophilic coating layer forms a continuous layer on the first surface of the impermeable substrate layer.

20. A disposable ultrasound transducer interface pad configured to adhere to at least one of an operational portion of an ultrasound transducer or a skin surface of a patient, the ultrasound transducer interface pad comprising:
a flexible, impermeable substrate layer having a first surface and a second surface opposite the first surface, the impermeable substrate layer having a thickness ranging from 0.025 millimeter (mm) to 1.00 mm;
a first hydrophilic coating layer applied to the first surface of the impermeable substrate layer, the first hydrophilic coating layer having a thickness ranging between 0.002 mm and 0.005 mm and activatable to provide an acoustic coupling interface between the ultrasound transducer and the patient; and
an adhesive layer applied to the second surface of the impermeable substrate layer and configured to adhere to the operational portion of the ultrasound transducer or the skin surface of the patient, the adhesive layer having a thickness ranging from 0.025 mm to 0.2 mm.

21. The disposable ultrasound transducer interface pad of claim 20, further comprising a second hydrophilic coating layer applied to the second surface of the impermeable substrate layer, the second hydrophilic coating layer activatable to provide an additional acoustic coupling interface between the ultrasound transducer and the patient.

22. The disposable ultrasound transducer interface pad of claim 20, wherein the first hydrophilic material comprises one of an ultraviolet (UV) light curable hydrophilic material or a heat curable hydrophilic material.

23. The disposable ultrasound transducer interface pad of claim 20, further comprising a removable release layer coupled to the adhesive layer, the release layer configured to be removed from the adhesive layer to expose the adhesive layer for adhering the disposable ultrasound transducer interface pad to the operational portion of the ultrasound transducer or the skin surface of the patient.

24. The disposable ultrasound transducer interface pad of claim 20, wherein the impermeable substrate layer is configured to prevent particulates greater than 27 nanometers from penetrating the impermeable substrate layer according to ASTM F1671/F1671M-22.

25. The disposable ultrasound transducer interface pad of claim 20, wherein the first hydrophilic coating layer forms a continuous layer on the first surface of the impermeable substrate layer.
